(19) United States
(12) Patent Application Publication
(10) Pub. No.: US 2024/0033212 A1
(43) Pub. Date: Feb. 1, 2024

(54) COMPOSITION FOR CLEANSING KERATIN MATERIALS

(71) Applicant: L'OREAL, Paris (FR)

(72) Inventors: Francis PRUCHE, AULNAY-SOUS-BOIS (FR); Lucie TOURNIER-COUTURIER, AULNAY-SOUS-BOIS (FR)

(21) Appl. No.: 18/265,540

(22) PCT Filed: Dec. 6, 2021
(86) PCT No.: PCT/EP2021/084451

§ 371 Date: Jun. 6, 2023

(30) Foreign Application Priority Data
Dec. 7, 2020 (FR) FR2012780

(51) Int. Cl. A61K 8/99; A61K 8/73; A61K 8/02; A61Q 5/02; A61Q 19/10; A61K 8/64

(52) U.S. Cl. CPC A61K 8/99; A61K 8/735; A61K 8/0216; A61Q 5/02; A61Q 19/10; A61K 8/64

(57) ABSTRACT

The present invention is targeted at a composition, in particular a cosmetic and non-therapeutic composition, for the care, the hygiene and/or the cleansing of keratin materials, comprising at least (i) all or part of a lysate, isolated or not, from a medium for the fermentation of a non-pathogenic and aerobic proteobacterium or a (glyco)oligopeptide fraction isolated from such a lysate, (ii) at least one mucopolysaccharide, in particular with a weight of greater than 10 kD, and (iii) at least one moisturizing agent distinct from (i) and from (ii), preferably in aqueous formulation and in the presence of electrolytes. It is additionally targeted at a method for care, hygiene and/or cleansing of a keratin material, comprising the use of such a composition.

### TECHNICAL FIELD

The present invention relates to the field of care, hygiene and/or cleansing compositions for the cosmetic treatment of keratin materials, in particular the skin, of the face and of the body, and keratin fibres, such as head hair and the beard.

It is more particularly targeted at providing a cleansing product for keratin materials, capable of effectively substituting for conventional aqueous cleansing over all or part of the body and which is also suitable for washing the hair.

### DISCLOSURE OF THE INVENTION

As emerges from what follows, the inventors have developed a novel body care, hygiene and/or cleansing product which is effective and which has the significant advantage of having been prepared from the viewpoint of the composition of the saliva, which is a very dilute fluid, more than 99% composed of water.

For the record, the saliva is composed of a variety of electrolytes, including sodium, potassium, calcium, magnesium, bicarbonate and phosphate ions. Immunoglobulins, proteins, enzymes, mucins and nitrogenous entities, such as urea and ammonia, are also present in the saliva. The biocarbonates, phosphates and urea act to modulate the pH and the buffering power of the saliva. The macromolecular proteins and the mucins serve to cleanse, to aggregate and/or to attach oral microorganisms and contribute to the metabolism of dental plaque. The calcium ions, phosphate ions and proteins modulate in particular demineralization and remineralization. With regard to the immunoglobulins, proteins and enzymes, they contribute to the antibacterial action. Finally, as seromucosal coating, the saliva lubricates and protects the buccal tissues.

The composition according to the invention is thus advantageously a “bio-inspired” product. As such, it makes it possible to meet the expectations of a large number of consumers who desire to be freed, henceforth as much as possible, from “chemical” products in favour of “natural” products which are advantageously not persistent in the environment. Contrary to all expectations, the inventors have furthermore found that the composition according to the invention provides benefits in terms of cleansing, moisturizing and lubrication while respecting the microbiome of keratin materials, in particular the skin and hair, and provides, on the one hand, a novel mild and pleasant sensory perception and, on the other hand, a protective effect for keratin materials by a film-forming effect after drying.

### SUMMARY OF THE INVENTION

More specifically, the invention is mainly targeted at a composition, in particular a cosmetic and non-therapeutic composition, for the care, the hygiene and/or the cleansing of keratin materials, comprising at least:
(i) all or part of a lysate, isolated or not, from a medium for the fermentation of a non-pathogenic and aerobic proteobacterium or a (glyco)oligopeptide fraction isolated from such a lysate,
(ii) at least one mucopolysaccharide, in particular with a weight of greater than 10 kD, (iii) at least one moisturizing agent distinct from (i) and from (ii) and in particular chosen from glycerol, aminoglycerol, urea, their salts and their mixtures,
   if appropriate, at least one electrolyte and,
if appropriate, at least one cosmetic active principle distinct from (i), (ii) and (iii).

According to an alternative embodiment, the composition contains a whole lysate or a lysate extract, isolated from its fermentation medium.

According to another alternative embodiment, the composition contains said lysate within a complete fermentation medium containing it.

In particular, the bacterium belongs to the Vitreoscilla sp. genus. More particularly, it is the bacterium Vitreoscilla filiformis and preferably the Vitreoscilla filiformis ATCC 15551 strain.

According to a particular embodiment, the present invention concerns a composition, in particular a cosmetic and non-therapeutic composition, for the care, the hygiene and/or the cleansing of keratin materials, comprising at least:
(i) all or part of a lysate, isolated or not, from a medium for the fermentation of a non-pathogenic and aerobic proteobacterium or a (glyco)oligopeptide fraction isolated from such a lysate,
(ii) at least one mucopolysaccharide with a weight of greater than 10 kD,
(iii) at least one moisturizing agent distinct from (i) and from (ii) and chosen from glycerol, aminoglycerol, urea, their salts and their mixtures,
   if appropriate, at least one electrolyte and,
if appropriate, at least one cosmetic active principle distinct from (i), (ii) and (iii).

According to another particular embodiment, the present invention concerns a composition, in particular a cosmetic and non-therapeutic composition, for the care, the hygiene and/or the cleansing of keratin materials, comprising at least:
(i) all or part of a lysate, isolated or not, from a medium for the fermentation of a non-pathogenic and aerobic proteobacterium or a (glyco)oligopeptide fraction isolated from such a lysate, said proteobacterium being the bacterium Vitreoscilla filiformis and preferably the Vitreoscilla filiformis ATCC 15551 strain,
(ii) at least one carrageenan or one of its salts with a weight of greater than 10 kD,
(iii) at least one moisturizing agent distinct from (i) and from (ii) and chosen from glycerol, aminoglycerol, urea, their salts and their mixtures,
   if appropriate, at least one electrolyte and,
if appropriate, at least one cosmetic active principle distinct from (i), (ii) and (iii).

According to another particular embodiment, the present invention concerns a composition, in particular a cosmetic and non-therapeutic composition, for the care, the hygiene and/or the cleansing of keratin materials, comprising at least:
(i) a (glyco)oligopeptide fraction isolated from a lysate from a medium for the fermentation of proteobacterium Vitreoscilla filiformis, in particular the Vitreoscilla filiformis ATCC 15551 strain,
(ii) at least one carrageenan or one of its salts with a weight of greater than 10 kD, (iii) at least one moisturizing agent distinct from (i) and from (ii) and chosen from glycerol, aminoglycerol, urea, their salts and their mixtures,
   if appropriate, at least one electrolyte and,
if appropriate, at least one cosmetic active principle distinct from (i), (ii) and (iii).

According to another particular embodiment, the present invention concerns a composition, in particular a cosmetic and non-therapeutic composition, for the care, the hygiene and/or the cleansing of keratin materials, comprising at least:
(i) a (glyco)oligopeptide fraction isolated from a lysate from a medium for the fermentation of proteobacterium Vitreoscilla filiformis, in particular the Vitreoscilla filiformis ATCC 15551 strain,
(ii) at least one carrageenan or one of its salts with a weight of greater than 10 kD,
(iii) at least one moisturizing agent distinct from (i) and from (ii) and which is glycerol,
   if appropriate, at least one electrolyte and,
if appropriate, at least one cosmetic active principle distinct from (i), (ii) and (iii).

According to one embodiment, the moisturizing agent is chosen from glycerol, aminoglycerol, urea, their salts and their mixtures.

According to another embodiment, the moisturizing agent comprises glycerol, aminoglycerol, urea, their salts and their mixtures.

According to another embodiment, the moisturizing agent consists in glycerol, aminoglycerol, urea, their salts and their mixtures.

A composition according to the invention is suitable for administration by the topical route, that is to say for application at the surface of keratin materials.

According to the invention, the term “keratin materials” is understood to mean the skin of the body, of the face and/or of the area around the eyes, the lips, the nails, the mucous membranes, the eyelashes, the eyebrows, body hair, the beard, the scalp and/or head hair, or any other cutaneous region of the body. More particularly, the keratin materials according to the invention are the scalp, head hair and/or the skin.

The term “the skin” is understood to mean all of the skin of the body, and preferably the skin of the face, of the neckline, of the neck, of the arms and forearms, indeed even more preferably still the skin of the face, in particular of the forehead, nose, cheeks, chin and area around the eyes.

According to a first alternative embodiment, the composition according to the invention comprises water, in particular in a proportion of at least 10% by weight, in particular more than 50% by weight and more particularly more than 80% by weight, with respect to its total weight, and at least one electrolyte and is thus provided in the form of an aqueous solution. Thus, the invention is in addition targeted at a non-therapeutic cosmetic method for the care, the hygiene and/or the cleansing of a keratin material, comprising the application, to said keratin material, of at least one composition according to the invention containing at least water and at least one electrolyte.

According to a second alternative embodiment, the composition according to the invention is in a solid and preferably anhydrous form.

Within the meaning of the invention, the term “anhydrous” is understood to describe a composition containing less than 10% by weight of water, in particular less than 5% by weight of water, in particular less than 2% by weight of water, with respect to its total weight, and preferably being completely devoid of water.

Within the meaning of the invention, a solid composition may or may not be in the pulverulent state.

More particularly, it can be provided in the form of a lyophilisate.

The present invention is then also targeted at a non-therapeutic cosmetic method for the care, the hygiene and/or the cleansing of a keratin material, comprising at least the stages consisting in:
(a) having available a physiologically acceptable aqueous medium,
(b) having available a composition according to the invention in a solid form, in particular an anhydrous solid form, and especially in the lyophilisate state,
(c) extemporaneous bringing of said composition (b) into contact with said aqueous medium (a) under conditions favourable to the dissolution and/or dispersion of the components of said composition in said medium, and
(d) bringing the mixture obtained in the preceding stage into contact with said keratin material,
it being understood that at least one electrolyte is present in said aqueous medium (a) and/or in said composition (b).

Within the meaning of the present invention, the term “physiologically acceptable medium” is understood to denote a medium which is suitable for the administration of a composition by the topical route and which is compatible with all the keratin materials of human beings, such as the skin, the lips, the nails, the mucous membranes, the eyelashes, the eyebrows, the scalp and/or the head hair, or any other cutaneous region of the body. According to the invention, a physiologically acceptable medium is preferentially a cosmetically acceptable medium, that is to say a medium without odour or unpleasant appearance and which is entirely compatible with the topical administration route.

Thus, a composition according to the invention containing water, and in particular in the form of an aqueous solution, can be used as product for cleansing the body, such as a shower product, a product for the bath, a product for cleansing the hands, as a product for cleansing head hair, such as a shampoo, or also for cleansing the face, for example as make-up removing product for the eyes, the face or the lips.

The present invention is also targeted at a non-therapeutic cosmetic method for scrubbing the skin, comprising the application, to said skin, of at least one cosmetic composition according to the invention containing at least water and at least one electrolyte.

It is also targeted at a non-therapeutic cosmetic method for removing make-up from the eyelashes, lips and/or skin, in particular of the face, comprising the application, to said region from which make-up is to be removed, of at least one cosmetic composition according to the invention containing at least water and at least one electrolyte.

### BRIEF DESCRIPTION OF THE DRAWINGS

A better understanding of the invention can be obtained on reading the detailed description which will follow, non-limiting implementational examples thereof, and on examining FIG. 1.

FIG. 1 schematically represents the body movement protocol for cleansing taken into consideration for testing the effectiveness of compositions according to the invention.

### DETAILED DESCRIPTION

### Composition According to the Invention

As specified above, the compositions according to the invention combine at least all or part of a lysate, isolated or not, from a medium for the fermentation of a proteobacterium or a specific fraction of such a lysate, at least one mucopolysaccharide and at least one ancillary moisturizing agent and are applied topically in a form combined with water and with one or more electrolytes.

Lysate from Medium for the Fermentation of Bacteria

A lysate commonly denotes a material obtained on conclusion of the destruction or dissolution of biological cells by a phenomenon termed cell lysis, thus bringing about the release of the intracellular and cellular biological constituents naturally contained in the biological cells under consideration.

Within the meaning of the present invention, the term lysate denotes all of the lysate obtained by lysis of the proteobacterium concerned. A lysate is thus formed of all of its intracellular biological constituents, in particular its metabolites and the constituents of the cell walls and membranes generated during its cell lysis.

Within the meaning of the invention, the term “metabolite” denotes any substance resulting from the metabolism of the microorganism under consideration according to the invention. This cell lysis can be accomplished by various technologies, such as, for example, osmotic shock, thermal shock, by ultrasound, or under mechanical stress of centrifugation type. More particularly, this lysate can be obtained according to the technology described in Patent U.S. Pat. No. 4,464,362.

As specified above, according to a specific embodiment, a composition according to the invention contains all or only a part of a lysate from the fermentation medium or the biomass of a proteobacterium. Thus, a part of the lysate can be isolated from the whole lysate by a centrifugation method. It can, for example, be the supernatant of the lysate.

Mention may in particular be made, by way of illustration and without limitation of the non-pathogenic and aerobic proteobacteria suitable for the invention, of the bacterium/bacteria belonging to the Vitreoscilla sp. genus, to the Sphingomonas sp. genus or to the Pseudoalteromonas sp. genus.

More specifically, the bacterium is a non-synthetic filamentous bacterium, as defined according to the classification of Bergey's Manual of Systematic Bacteriology (Vol. 3, sections 22 and 23, 9th edition, 1989), belonging to the Vitreoscilla sp. genus. More particularly, it is the bacterium Vitreoscilla filiformis.

In a specific embodiment, the bacterium is a Vitreoscilla filiformis strain, preferably the Vitreoscilla filiformis ATCC 15551 strain. In order to prepare the lysate of a bacterium of the Vitreoscilla sp. genus, reference may be made in particular to the description of Patent Application WO-A-94-02158 or of Patent Application WO2008/138839.

According to another specific alternative form, the composition according to the invention contains the lysate in a complete fermentation medium.

Within the meaning of the invention, the expression “lysate in a complete fermentation medium” means that the lysate is present, in the composition according to the invention containing it, formulated in its original complete culture medium, which complete culture medium is the medium in which the bacteria were cultured until after the microbial growth phase which led to the use of the nutritive substrates initially present in the culture medium. More specifically, this “complete fermentation medium” is understood to denote a medium resulting from the culture method which has been used for the growth and the cell lysis of the microorganism, said medium moreover not having been subjected to any additional handling targeted at separating and/or removing all or part of its non-aqueous constituents. More particularly, the complete fermentation medium is all or part, in terms of amount, of the culture medium which served for the fermentation of said bacterium and in which its cell lysis was consecutively carried out. This “complete” fermentation medium thus contains the cytoplasmic and cytosolic fractions, the cell wall fragments and the metabolites formed and/or released during the cell lysis of said bacterium and all of the biological entities capable of being generated and released spontaneously by the bacterium during its fermentation process and thus already present in the fermentation medium before the cell lysis of the latter.

Thus, in a specific embodiment, a composition according to the invention contains the lysate of the Vitreoscilla filiformis strain, preferably the Vitreoscilla filiformis ATCC 15551 strain, in its complete fermentation medium. This lysate in complete medium can in particular be obtained according to the protocol described in Patent Application EP 2 830 588.

According to yet another specific alternative form, a composition according to the invention contains the (glyco)oligopeptide fraction isolated from a lysate of a biomass referred to also as medium for fermentation of a proteobacterium.

Within the meaning of the invention, the expression (glyco)oligopeptide fraction covers a fraction containing at least one oligopeptide and/or at least one glycooligopeptide.

Within the meaning of the present invention, oligopeptide is understood to mean a heterooligopeptide and glycooligopeptide is understood to mean an oligopeptide covalently bonded to a mono- or oligocarbohydrate glycosyl radical, which is generally small in size. The weight of the glycosyl part of a glycooligopeptide can range from 5% to 40% of the molecular weight of the glycooligopeptide assembly.

This oligopeptide fraction can be obtained via a method consisting in:
the culturing of at least one proteobacterium on a fermentation medium under conditions conducive to the proliferation of said bacterium, and
a centrifugation stage, and
optionally adjustment of the pH, and
the enzymatic digestion of the residue derived from the centrifugation of said bacteria in said fermentation medium.

The isolated (glyco)peptide fraction is thus obtained by a stage of enzymatic digestion of said lysate carried out starting from carbohydrase and protease enzymes.

More specifically, the oligopeptide fraction within the meaning of the invention in particular denotes the fraction resulting from the digestion of the residue resulting from the centrifugation, which is optionally adjusted to a given pH and subsequently treated by a carbohydrase enzyme and a protease enzyme.

In order to isolate the oligopeptide fraction of a proteobacterium/bacteria of a Vitreoscilla filiformis strain, reference may be made in particular to the description of Patent Application WO2018/108973. More specifically, the oligopeptide fraction thus isolated is constituted of 30% to 50% of oligopeptides, between 1% and 10% of amino acids and of 10% to 20% of a mixture of oligosaccharides and of monosaccharides. Generally, the oligopeptides have a molecular weight of between 200 Da and 3 kDa, with a peptide distribution centered on the 200-800 Da region. In particular, the oligopeptides are peptides between 2 and 7 amino acids on average. Glutamic acid, glutamine, histidine and arginine constitute at least 15% of said 1% to 10% of amino acids. In a preferred way, the oligosaccharides are chosen from stachyose, sucrose, glucose, fructose and trehalose.

Mucopolysaccharide

Mucopolysaccharides are a class of polysaccharides, also called glycosaminoglycans, composed of aminosugars chemically bonded as repeat units to form compounds of unbranched linear polymer type.

These compounds are very particularly advantageous for contributing long-lasting moisturizing to the skin or to the scalp.

In particular, mucopolysaccharides are present in the compositions according to the invention in a form distinct from a lysate.

Mucopolysaccharides with weights (that is to say molecular weights) of greater than 10 kD are more particularly suitable for the invention.

The mucopolysaccharides suitable for the invention can in particular be chosen from polysaccharides extracted from marine algae, in particular green, red or brown algae, and very particularly from carrageenans, alginates, ulvans, fucoidans, chitosan, chitin, their salts, their extracts and their mixtures.

A composition according to the invention can thus contain, as mucopolysaccharides, at least one alginate.

Alginic acid is a natural substance extracted from brown algae or from bacteria. Alginic acid forms water-soluble salts (alginates) with alkali metals or alkaline earth metals, such as sodium, calcium, potassium or lithium, and (C₁-C₈)alkylamines and (C₁-C₈)alkylammoniums, such as methylamine, ethanolamine, diethanolamine and triethanolamine.

A composition according to the invention can also contain, as mucopolysaccharides, at least one ulvan, one of its salts or extracts. Ulvans are sulfated anionic polysaccharides containing rhamnose which are obtained from green algae belonging to the order of the Ulvales. Ulvans are water-soluble sulfated anionic polysaccharides which are located in the cell wall. The ulvan suitable for the invention preferably originates from Ulva lactuca. The ulvan can be employed in the form of the extract called “OligoUlvan” and which can be obtained according to Patent FR 3 075 644.

A composition according to the invention can also contain, as mucopolysaccharides, at least one fucoidan, one of its salts or extracts. Fucoidan, an algal extract, is a generic term denoting a polysaccharide which comprises sulfated fucose as saccharide component. It can in particular be the commercial product Matrigenics distributed by Codif.

A composition according to the invention can also contain, as mucopolysaccharides, at least one (co)polymer of glucosamine chosen from chitosan and its salts. Chitosan is a polysaccharide composed of D-glucosamine and of N-acetyl-D-glucosamine. It can be employed as is or in the form of one of its salts, such as salts with a carboxylic acid comprising from 1 to 6 carbon atoms, such as acetic acid, thioglycolic acid, adipic acid, ascorbic acid, formic acid, glycolic acid and lactic acid. This chitosan can in particular be employed in the form of the commercial product Kionutrime distributed by Kytozyme.

In particular, a composition according to the invention contains, as mucopolysaccharides, at least one carrageenan or one of its salts.

Carrageenans are sulfated and linear polysaccharides, comprising long galactan chains formed of disaccharide units, which constitute the cell walls of varied red algae, from which they can be obtained. Mention may be made, among these red algae, in a non-limiting way, of Kappaphycus alvarezii, Eucheuma denticulatum, Eucheuma spinosum, Chondrus crispus, Betaphycus gelatinum, Gigartina skottsbergii, Gigartina canaliculata, Sarcothalia crispata, Mazzaella musceapellata, Mastnocariformatus cordiforme, Irocariformata cordiforme, Irrocariformata cordiforme, Mazzaella musceapusellatus and Irocariformatus cordiforme. The carrageenans which are particularly suitable for the invention are the carrageenans of lambda, kappa or iota form, their hybrids and their mixtures in all proportions. Thus, a single carrageenan or a mixture carrageenans may be concerned.

More specifically, these carrageenans are chosen from the following compounds:
Iota carrageenan;
Kappa carrageenan;
Lambda/kappa carrageenan mixture, in particular sold under the name Satiagel, and
Lambda carrageenan.

The carrageenans of the present invention can be used in the acid form or in the salified form. The acceptable salts which can be mentioned in a non-limiting way comprise the lithium, sodium, potassium, calcium, zinc and ammonium salts or the salts obtained with an organic base counterion, such as a primary, secondary or tertiary (C₁-C₆)alkylamine, in particular triethylamine or butylamine, indeed even the basic form of amino acids, such as arginine and lysine, for example.

The molecular weight of the carrageenans of use for the present invention is generally between 10×10³ and 100×10⁶ Da and in particular between 15×10³ and 10×10⁵ Da. The carrageenans which can be used in particular in the invention predominantly comprise lambda forms or are provided in the lambda form. The term “predominantly” means that the percentage of this type of chain in the composition of the product is greater than or equal to 50%, it being possible for this proportion to be greater than or equal to 80% in some embodiments.

In particular, a composition according to the invention contains a carrageenan derived from Chondrus crispus or Kappaphycus alvarezii.

The carrageenans extracted from Chondrus crispus can in particular be those sold by Cargill under the respective names Satiagum UTC 30 Carrageenan lamda Cargill and Satiagum UTC 10 Carrageenan lamda Cargill.

A composition according to the invention, in aqueous presentation formulation, can comprise from 0.01% to 5% and preferably from 0.02% to 0.1% by weight of mucopolysaccharide(s) and in particular of carrageenan(s), with respect to the total weight of the composition.

The mucopolysaccharide(s) according to the invention is (are) combined with at least one ancillary moisturizing agent in particular chosen from glycerol, aminoglycerol, urea, their salts and their mixtures.

The moisturizing agent is described as ancillary as it is distinct from a lysate or from a mucopolysaccharide as required according to the invention and described in detail above.

In particular, a composition according to the invention comprises one or more mucopolysaccharide(s) and one or more ancillary moisturizing agents especially in a moisturizing agent(s)/mucopolysaccharide(s) ratio by weight varying from 50 to 1000 and preferably from 100 to 500 and in particular from 200 to 400.

The amount of ancillary moisturizing agent(s) in a composition according to the invention, in aqueous formulation, can vary from 1% to 30%, preferably from 5% to 20%, by weight, with respect to the total weight of the composition.

Electrolytes

A composition according to the invention can additionally comprise one or more electrolytes.

Mention may more particularly be made, as electrolytes which can be used in the composition according to the invention, of alkaline earth metal salts and in particular calcium salts, alkali metal salts and, for example, sodium and potassium salts, and also magnesium or manganese salts, and their mixtures.

The ions constituting these salts can be chosen, for example, from carbonates, bicarbonates, sulfates, phosphates, sulfonates, glycerophosphates, borates, bromides, chlorides, fluorides, nitrates, acetates, hydroxides or persulfates and also ions of α-hydroxy acids (citrates, tartrates, lactates, malates) or of fruit acids, ions of β-hydroxy acids (salicylates, 2-hydroxyalkanoates, n-alkylsalicylates and n-alkanoylsalicylates) or also ions of amino acids (aspartate, arginate, glycocholate, fumarate).

Generally, a composition according to the invention contains at least a mixture of several electrolytes chosen from alkaline earth metal salts and in particular calcium salts, alkali metal salts and in particular sodium and potassium salts, and also magnesium or manganese salts and carbonates, bicarbonates, sulfates, phosphates, bromides, chlorides and fluorides. Use may also be made of a natural mixture of electrolytes or of a mixture, the composition of which is close to that of a natural mixture, and in particular the powder mixtures sold under the trade names powder from Bulk Powders® and electrolytes from Global Medics®, to be diluted in demineralized water.

The electrolytes can be introduced as is or in the form of a solution generally water or an aqueous medium containing them in the solute state, such as, for example, a seawater, a thermal water or a mineral water. In general, a mineral water is fit for consumption, which is not always the case for a thermal water. Each of these waters contains, inter alia, dissolved minerals and trace elements.

The nature of its salts and their respective amounts are, of course, adjusted in order to be physiologically acceptable. Thus, for example, in the case where seawater is considered as source of electrolytes, it is employed in a form depleted in NaCl, indeed even desalified.

Aqueous Medium

A composition according to the invention can comprise at least 50% by weight and in particular at least 80% by weight of an aqueous medium, with respect to its total weight.

In particular, a composition according to the invention comprises water and at least one electrolyte and in particular is in the form of an aqueous solution.

This aqueous medium is or contains water and preferably spring, thermal or mineral water. It can also be formed in part of seawater, which, as described in detail above, can be considered in a composition according to the invention as source of electrolytes.

The thermal water or the mineral water used can be chosen, for example, from Vittel water, Vichy basin water, Uriage water, La Roche-Posay water, La Bourboule water, Enghien-les-Bains water, Saint-Gervais-les-Bains water, Néris-les-Bains water, Allevard-les-Bains water, Digne water, Maizières water, Neyrac-les-Bains water, Lons-le-Saunier water, Eaux-Bonnes water, Rochefort water, Saint Christau water, Les Fumades water, Avéne water and Tercis-les-Bains water.

According to one embodiment, a composition according to the invention contains water and at least a mixture of several electrolytes chosen from alkaline earth metal salts and in particular calcium salts, alkali metal salts and in particular sodium and potassium salts, and also magnesium or manganese salts and carbonates, bicarbonates, sulfates, phosphates, bromides, chlorides and fluorides, in particular in a proportion of 0.1% to 20%, preferably of 0.10% to 0.5%, with respect to its total weight.

According to a specific alternative form of the invention, the composition according to the invention contains:
at least the oligopeptide fraction isolated from a lysate from a medium for the fermentation of a proteobacterium Vitreoscilla filiformis, in particular the Vitreoscilla filiformis ATCC 15551 strain,
at least one mucopolysaccharide, in particular at least one carrageenan,
at least one moisturizing agent chosen from glycerol, aminoglycerol, urea, their salts and their mixtures,
at least desalified seawater, and
water.

Mention may in particular be made, by way of illustration and without limitation of compositions according to the invention, of the aqueous care, hygiene and/or cleansing compositions containing:
at least the oligopeptide fraction isolated from a lysate from a medium for the fermentation of a proteobacterium Vitreoscilla filiformis, in particular the Vitreoscilla filiformis ATCC 15551 strain,
approximately 0.05% by weight of at least one mucopolysaccharide, in particular at least one carrageenan, with respect to its total weight,
approximately 15% by weight of at least one moisturizing agent chosen from glycerol, aminoglycerol, urea, their salts and their mixtures, with respect to its total weight,
approximately 10% by weight of desalified seawater, with respect to its total weight, and
approximately 74% by weight of water, distinct from the seawater, with respect to its total weight.

Surfactants A composition according to the invention can additionally contain one or more surfactants, in particular for their detergent properties.

This surface-active component is, of course, chosen, on the one hand, for its compatibility with the other ingredients present in the composition and, on the other hand, in order not to prejudice the qualities desired for the claimed composition.

In particular, this surface-active component is chosen from biosurfactants.

Surfactants of lauryl sulfate type may also be suitable. However, when the compositions according to the invention are very particularly dedicated to being comparable to “biobased” products, biosurfactants are favoured.

For the record, biosurfactants are surfactants of microbial origin. They are produced and excreted by several specific bacterial strains, in particular those of the Pseudomonas and Bacillus genera.

Several types of biosurfactants exist, including glycolipids (including rhamnolipids) and lipopeptides very particularly suitable for the invention.

These biosurfactants have the advantage both of possessing an effectiveness as surfactant and of being biodegradable and thus of being advantageously non-persistent in the environment, one of the objectives pursued according to the present invention. Such surfactants are in particular described in the paper by D. K. De Santos et al., 18 Mar. 2016, International Journal of Molecular Sciences.

Mention may in particular be made, by way of illustration and without limitation of the surfactants suitable for the invention, of spiculisporic acid (S acid) and its salts, in particular that sold under the name S acid by Iwota Chemical, rhamnolipids, in particular that sold under the name Rhamnolipid L by Kaneka, sophorolipids, in particular that sold under the name Sopholiance S by Soliance-Givaudan, and surfactins, in particular those sold under the trade name Surfactin Sodium Salt by Evonik.

Adjuvants

In a known way, care and cleansing compositions and thus those according to the invention can contain, besides the compounds required according to the invention, one or more adjuvants conventional in the cosmetics field (fragrance, fillers, odour absorbers, colouring materials, and the like) and one or more cosmetic or therapeutic active principles other than the active principles required according to the invention.

More particularly, this or these other cosmetic or therapeutic active principles can be chosen from sunscreens; desquamating or exfoliating agents, such as, for example, compounds derived from salicylic acid or their salts; depigmenting agents; propigmenting agents; α-hydroxy acids; antibacterial agents; agents for combating free radicals; agents for combating pollution; retinoids; extracts of fungi, of plants other than algae or of bacteria other than proteobacteria; hydrolysed, partially hydrolysed or nonhydrolysed proteins; enzymes, hormones, yeast extracts, vitamins and their derivatives, flavonoids and isoflavones, and their mixtures.

They can in particular be chosen from enzymes, in particular proteases of use for the digestion of corneocytes, such as, for example, papain from BASF or Patel Papain Industries, β-glucosidases, such as those of almonds, such as cellobiose sold by Novozymes or Aspergillus niger β-glucosidase from Megazymes, subtilisin, in particular such as those sold by Novozyme, alcalase, such as in particular collupulin sold by DSM and bromelain from Enzybel.

The amounts of these various adjuvants are those conventionally used in the field under consideration and, for example, can vary from 0.01% to 5% and preferably from 0.1% to 1% by weight of the total weight of the composition.

Presentation Formulations

The compositions according to the invention are suitable for application by the external topical route. To do this, they can be provided at the time of the application in a liquid presentation form and in particular in the form of aqueous or aqueous/alcoholic solutions, of solutions or dispersions of the lotion, gel or serum type, indeed even of emulsions of liquid consistency. When the composition is an emulsion, the proportion of the fatty phase can range from 0.5% to 30% by weight and preferably from 1% to 20% by weight, with respect to the total weight of the composition. The oils, the waxes or the emulsifiers, indeed even coemulsifiers, used in the composition in the emulsion form are chosen from those conventionally used in the cosmetics field.

These liquid forms can, for example, be impregnated on an application substrate, for example a fibrous application substrate, such as a cotton swab, for example, or also of sponge type, in order to be subsequently applied to keratin materials, such as the skin of the face or of the body or head hair or the beard.

According to another alternative embodiment which is preferred, these liquid presentation forms can also be prepared extemporaneously at the very moment of the application, starting from a composition according to the invention in solid and preferably anhydrous form, in particular as described in detail above.

This solid presentation form can in particular consist of a lyophilised form which can be provided under the appearance of a tablet or oral lyophilisate and it is used in combination with a physiologically acceptable medium which is provided in a fluid form favourable to the dissolution and/or the dispersion of the tablet or oral lyophilisate. As specified above, this physiologically acceptable medium can contain all or part of the electrolyte component of the composition according to the invention.

This type of formulation in the solid and in particular lyophilisate form has the advantage(s) of being easily transportable because of greatly reduced bulk, high availability, easy use and not raising any difficulty and/or constraint in terms of storage.

This lyophilisation can be carried out according to conventional methods.

Thus, it can be advantageous for the composition in solid form to additionally contain a lyophilisation additive, in particular for facilitating the lyophilisation via the texturing of the composition, but also the rehydration of the lyophilised form, when the latter is provided in the powder, cake, tablet or film form. Mention may be made, by way of example of lyophilisation additive, of silica and its derivatives, clays, cellulose derivatives (HEC, HPC, HMPC, and the like), polymers of natural origin xanthans, locust bean gum, guar gums, pectins, agar, polymers of bacterial origin, such as hyaluronic acid, dextran, gellan and hydrogels, such as carbomers or AMPS derivatives.

It can also be advantageous for a composition according to the invention, dedicated to being dispersed in an aqueous medium, to contain a citric acid and sodium bicarbonate mixture which, by producing gas, facilitates its dissolution.

In an alternative form, the presentation formulation of such a composition of tablet or oral lyophilisate type can be adapted in order for the dispersing of the whole of this composition in a fixed amount of physiologically acceptable medium to provide a dilute composition dedicated to a single use.

According to a specific embodiment, the method according to the invention can comprise a stage consisting in dissolving and/or dispersing the composition of the invention in solid form in the water of a bath or of a shower or of a thermal water thus intended to be brought into contact with a keratin material and in particular the skin.

The contacting operation on the solid form can be carried out extemporaneously by its direct immersion in the water of a bath, for example. It is also possible to envisage carrying out this contacting operation more gradually. Thus, there exist shower head devices constructed to make possible the insertion into the head of solid compositions dedicated, for example, to care. The dissolution of the composition is then assured gradually on contact with the water diffusing through the head.

The mixture of composition in solid form and of the physiologically acceptable medium can be applied by any means making possible uniform distribution and in particular using a cotton swab, a rod, a brush, a gauze, a spatula or a pad, or also by spraying, and may or may not be removed by rinsing with water or using a mild detergent.

These compositions are prepared according to the usual methods.

The compositions according to the invention can be used on all keratin materials, such as the skin, the scalp, head hair, the eyelashes, the eyebrows, the nails or the mucous membranes, in particular in the form of a hygiene product, for example in the form of a product for cleansing the skin, mucous membranes and/or head hair or the beard, in particular in the form of a product for cleansing and/or removing make-up from the skin (of the face and/or of the body), in the form of a shower product, in the form of a shampoo and conditioner, in the form of a shaving product, in the form of a rinse-off mask, in the form of an exfoliating product (also called desquamating or deep cleansing agent), both for the face and for the body or for the hands, after addition of exfoliating particles.

Another subject-matter of the invention is a method for cleansing a keratin material, such as the skin, scalp, head hair, eyelashes, eyebrows, nails or mucous membranes, comprising at least one stage of application, to the keratin material, of a composition as defined above and, if appropriate, at least one rinsing stage.

The examples which follow serve to illustrate the invention without, however, exhibiting a limiting nature. The amounts indicated are as % by weight, unless otherwise mentioned, and are expressed as weight of solids. The names of the compounds used are shown as CTFA name or as chemical name.

Materials and Methods

Test of the Effectiveness of a Composition According to the Invention

This test is targeted at evaluating its cleansing power on a stain formed of a mixture of charcoal particles and sebum described in detail below and which is deposited on a skin model.

Skin Models:

Smooth synthetic skin (punch diameter 5 cm) from Biolax: Bioskin Plate K520 thickness 2 mm attached with double-sided Scotch tape to a cork sheet covered with aluminium paper.

Application

7.6 μl/cm 2 of a mixture of avocado oil (from Expanscience, virgin oil)+charcoal (Sigma-Aldrich, active charcoal) at 2% by weight of the mixture is applied, i.e. 5×30 μl of the solution (oil+2% charcoal), with the syringe pipette with inclination of the synthetic skin in order to distribute the solution homogeneously at ambient temperature and ambient humidity.

Cleansing Protocol

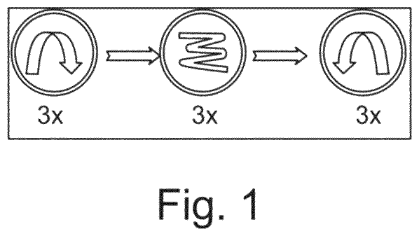

It is carried out 5 minutes after application of the staining composition. 2 ml of the washing solution considered are applied to a commercial make-up-removing cotton swab (Demakup), then a body movement for recovery/rubbing with the cotton swab is carried out over the region to be cleansed, in the form of 9 passes, with 3 fingers, 3 passes to the left 3 zigzag 3 towards the right, according to the protocol represented schematically in FIG. 1.

The effectiveness of the cleansing is assessed by visual observation of the state of the skin and of the state of the cotton swab. This effectiveness is characterized on a performance scale ranging from +++++ (the state of the skin observed is clean and perfectly decent) to + (the state of the skin is not clean and the stain is still observed).

The amount of stain recovered during the cleansing operation is determined according to the following protocol: the cotton swab is weighed before and after cleansing and the amount of stain recovered is estimated by subtraction of the weight of the cotton swab before cleansing from the weight of the cotton swab after cleansing.

Determination of the Sensory Experience

This sensory experience is described by assessing the greasy aspect experienced to the touch 3 minutes after the cleansing of the skin with the washing solution considered. The expert grades the intensity of the sensation noticed on a scale ranging from +++++ (pleasant sensory effect noticed because of non-greasy feel) to—(unpleasant sensory effect because of greasy feel).

### Example 1

Preparation of an Oligopeptide Fraction Isolated from a V. filiformis Lysate

The composition of the complete nutrient medium for the culturing of V. filiformis is described in detail in Table 1.

TABLE 1

| Chemical name | Concentration |
|---|---|
| Autolytic yeast extract | 4 g/l |
| Soybean papain peptone F | 3 g/l |
| Glucose-Roferose | 3 g/l |
| KH₂PO₄ | 0.088 g/l |
| CaCl₂ | 0.050 g/l |
| CuSO₄•5H₂O | 60 μg/l |
| MnSO₄•1H₂O | 152 μg/l |
| KI | 20 μg/l |
| ZnSO₄•7H₂O | 200 μg/l |
| AlCl₃•6H₂O | 100 μg/l |
| Deionized water | qsp 1 litre |

In order to obtain the oligopeptide fraction, the procedure as described below is carried out. The initial strain of V. filiformis is obtained from the ATCC (strain 15551).

The biomass is obtained by fermentation in an industrial bioreactor of 3000 effective litres; the composition of the culture medium is given above. During the culturing, the pH is kept constant (7.00), as is the temperature (26° C.) and the dissolved oxygen (0.5%). The culturing is stopped when the solids content reaches 0.6% and the glucose content reaches 0.035%.

The V. filiformis strain is fermented in its complete culture medium.

Everything is centrifuged and the residue is treated by enzymatic digestion.

For the enzymatic digestion, first a Viscozyme® carbohydrase sold by Novozyme is introduced in an amount of 0.2% w/w over a period of time of 3 h at a temperature of 55° C., the pH is adjusted to and maintained at 4.5 by citric acid, then a Sumizyme AP protease sold by Novozyme is introduced in an amount of 0.04% w/w over a period of time of 3 h at a temperature of 55° C. and the pH is maintained at 4.5 after adjustment by addition of citric acid, if necessary.

Subsequent to this second enzymatic digestion, the medium is neutralized to pH 7 using a sodium hydroxide (NaOH) solution. The enzyme is inactivated by heating the medium at a temperature of 90° C. for 15 minutes.

The neutralized and inactivated medium is clarified by successive filtrations, first with a 10 μm bag filter and then a K2 filter.

A stage of spray drying the clarified complete reaction medium is carried out with a Buchi mini spray dryer B-290 with the following parameters: Pump 30%; Inlet temperature 160° C.; Outlet temperature 85° C.; Aspirator 95; Q flow 35. The oligopeptide fraction is subsequently recovered in the powder form and is used as is in the examples which follow.

### Example 2

Preparation of a Composition in Accordance with the Invention

A formula A is prepared by simple mixing under a vortex at ambient temperature, that is to say varying from 18° C. to 25° C., of the 3 ingredients described in detail in Table 2.

TABLE 2

| Ingredients | Amount (g) |
|---|---|
| Glycerol | 300 |
| Millipore distilled | 500 |
| water (miliQ system) | |
| Seawater | 200 |
| (Holoslife ® or Rioka ®) | |

A formula B is prepared by heating the water to 70° C. and by incorporating in it, with stirring, the two other ingredients described in detail in Table 3, until they have dissolved.

TABLE 3

| Ingredients | Amount (g) |
|---|---|
| Carrageenan | 1 |
| (Satiagel from Cargill) | |
| Oligopeptide fraction of | 3 |
| Example 1 | |
| Millipore distilled water | 1000 |
| (miliQ system) | |

The two formulae are subsequently mixed to form the expected composition.

### Example 3

Preparation of Compositions Suitable for the Invention

The compositions 2a to 2f described in detail below in Table 4 were prepared according to the protocol described in detail in Example 2.

TABLE 4

| | Compositions (in g) | | | | | |
|---|---|---|---|---|---|---|
| Compounds | 2a | 2b | 2c | 2d | 2e | 2f |
| Glycerol | 150 | 150 | 150 | 150 | 150 | 150 |
| Carrageenan | 0.5 | 0.5 | 0.5 | 0.5 | 0.5 | 0.5 |
| (Satiagel from Cargill) | | | | | | |
| Oligopeptide fraction | 1.5 | 1.5 | 1.5 | 1.5 | 1.5 | 1.5 |
| of Example 1 | | | | | | |
| Papain (sold by BASF) | | 0.1 | | | | |
| Yeast extract (sold by | | | 0.1 | | | |
| Solabia) | | | | | | |
| Rhamnolipid (sold by Evonik) | | | | 0.1 | | |
| Lysine salt of 2-hydroxy-6- | | | | | 0.1 | |
| pentadecylbenzoic acid (as | | | | | | |
| described in detail in | | | | | | |
| WO201666811) | | | | | | |
| Canarium Luzonicum Gum | | | | | | 0.1 |
| Nonvolatiles (Elemi resin | | | | | | |
| from Firmenich) | | | | | | |
| NaCl-free seawater | 100 | 100 | 100 | 100 | 100 | 100 |
| (Holoslife ® or Rioka ®) | | | | | | |
| Millipore distilled water | 750 | 750 | 750 | 750 | 750 | 750 |
| (miliQ system) | | | | | | |
| pH | 6.5 | 6.8 | 6.5 | 6.4 | 6.6 | 6.6 |

### Example 4

Test of the effectiveness of cleansing of a composition according to the invention The composition 2a of Example 3 is tested for its effectiveness of cleansing and the qualities noticed by the user according to the protocols described in detail in the Materials and methods section.

The control micellar solution Créaline H2O (Bioderma) and water are tested in parallel as control washing solutions.

The control micellar solution Créaline H2O (Bioderma) comprises the following components: water, PEG-6 caprylic/capric triglycerides, fructooligosaccharides, mannitol, xylitol, rhamnose, cucumber fruit extract, PEG, cetrimonium bromide, EDTA.

Each test is carried out three times in order to evaluate the amount of stain recovered. The results obtained are reported in Table 5.

TABLE 5

| | | | Cotton | Cotton | |
|---|---|---|---|---|---|
| | | | swab + | swab + | |
| | | Dry | composition | control | Cotton |
| | | cotton | 2a of | micellar | swab + |
| Quality tested | | swab | Example 3 | composition | water |
| Effectiveness of the | | + | +++ | +++ | ++ |
| cleansing | | | | | |
| Amount of | Test 1 | 61 | 137 | 107 | 91 |
| stain | | | | | |
| recovered | | | | | |
| during the | Test 2 | 69 | 128 | 101 | 97 |
| cleansing | | | | | |
| operation | Test 3 | 76 | 139 | 105 | 95 |
| (mg) | | | | | |

| | | | | |
|---|---|---|---|---|
| Sensory experience | + | ++++ | ++ | − |
| (absence of greasy effect). | | | | |

It emerges from these results that the composition 2a of Example 3 according to the invention provides the best performance qualities in terms of effectiveness of cleansing and of sensory quality.

1-20. (canceled)

21. A composition for the care of keratin materials, comprising:
(i) all or part of at least one lysate derived from at least one non-pathogenic, aerobic proteobacterium fermentation medium, optionally wherein the composition comprises a (glyco)oligopeptide fraction isolated from the at least one lysate;
(ii) at least one mucopolysaccharide;
(iii) at least one moisturizing agent distinct from (i) and from (ii);
(iv) optionally, at least one electrolyte; and
(v) optionally, at least one cosmetically active ingredient distinct from (i)-(vi).

22. The composition according to claim 21, wherein the at least one lysate is isolated from the fermentation medium.

23. The composition according to claim 21, wherein the at least one lysate is a whole lystate.

24. The composition according to claim 21, wherein the at least one lysate is a part of a lysate.

25. The composition according to claim 22, comprising a whole lysate in a complete fermentation medium.

26. The composition according to claim 22, wherein the isolated (glyco)peptide fraction is obtained by enzymatic digestion of the at least one lysate using at least one carbohydrase, at least one protease, or combinations of two or more thereof.

27. The composition according to claim 21, wherein the bacterium is a Vitreoscilla filiformis strain.

28. The composition according to claim 21, wherein the bacterium is the Vitreoscilla filiformis (ATCC 15551) strain.

29. The composition according to claim 21, comprising at least one mucopolysaccharide extracted from marine algae.

30. The composition according to claim 21, wherein the weight ratio of the total amount of the moisturizing agent(s) to the total amount of the mucopolysaccharide(s) ranges from 50:1 to 1000:1.

31. The composition according to claim 21, further comprising a mixture of electrolytes chosen from 1) alkaline earth metal salts; or 2) magnesium or manganese salts, carbonates, bicarbonates, sulfates, phosphates, bromides, chlorides or fluorides.

32. The composition according to claim 21, wherein the composition is in a solid form.

33. The composition according to claim 21, further comprising water and at least one electrolyte.

34. The composition according to claim 33, further comprising an aqueous medium in an amount of at least 50% by weight, relative to the total weight of the composition.

35. The composition according to claim 34, wherein the total amount of munopolycaccharide(s) ranges from 0.01% to 5% by weight, relative to the total weight of the composition comprising the aqueous medium.

36. The composition according to claim 34, comprising:
(i) at least one oligopeptide fraction isolated from at least one lysate, wherein the at least one lysate is derived from at least one Vitreoscilla filiformis fermentation medium,
(ii) at least one mucopolysaccharide in an amount ranging from 0.02% to by weight, relative to the total weight of the composition;
(iii) at least one moisturizing agent chosen from glycerol, aminoglycerol, urea, their salts thereof, or mixtures of two or more thereof wherein the total amount (iii) moisturizing agent(s) ranges from 5% to 20%, relative to the total weight of the composition;
desalified seawater; and
water, distinct from the seawater, in an amount of at least 10% by weight, relative to the total weight of the composition.

37. A method for the care of keratin materials, comprising:
a) dissolving a composition for the care of keratin materials in a physiologically acceptable aqueous solution, wherein the composition for the care of keratin materials comprises:
(i) all or part of at least one lysate derived from at least one non-pathogenic, aerobic proteobacterium fermentation medium, optionally wherein the composition comprises a (glyco)oligopeptide fraction isolated from the at least one lysate;
(ii) at least one mucopolysaccharide;
(iii) at least one moisturizing agent distinct from (i) and from (ii);
(iv) optionally, at least one electrolyte; and
(v) optionally, at least one cosmetically active ingredient distinct from (i)-(vi).
b) applying dissolved composition a) to the keratin materials wherein at least one electrolyte is present in the dissolved composition.

38. The method according to claim 37, wherein the method further comprises c) cleansing the keratin materials, and wherein the composition further comprises water and at least one electrolyte.

39. The method according to claim 37, wherein the method further comprises c) scrubbing the keratin materials, and wherein the composition further comprises water and at least one electrolyte.

40. The method according to claim 37, wherein the method further comprises c) removing make-up from the keratin materials, and wherein the composition further comprises water and at least one electrolyte.
